(19) United States
(12) Patent Application Publication
(10) Pub. No.: US 2023/0312508 A1
(43) Pub. Date: Oct. 5, 2023

(54) IMPROVED PROCESS FOR PREPARATION OF ANTHRANILAMIDES

(71) Applicant: GSP CROP SCIENCE PRIVATE LIMITED, Navrangpura, Ahmedabad (IN)

(72) Inventors: Avinash Sheshrao MANE, Navrangpura, Ahmedabad (IN); Nilesh N. JANI, Navrangpura, Ahmedabad (IN); Kenal V. SHAH, Navrangpura, Ahmedabad (IN); Bhavesh V. SHAH, Navrangpura, Ahmedabad (IN); Nikulsinh SERVAIYA, Navrangpura, Ahmedabad (IN); Ritesh PATEL, Navrangpura, Ahmedabad (IN)

(73) Assignee: GSP CROP SCIENCE PRIVATE LIMITED, Navrangpura, Ahmedabad (IN)

(21) Appl. No.: 18/245,636

(22) PCT Filed: Feb. 5, 2021
(86) PCT No.: PCT/IB2021/050935

§ 371 Date: Mar. 16, 2023

(30) Foreign Application Priority Data
Dec. 24, 2020 (IN) 202021056370

(51) Int. Cl. C07D 401/04; A01P 7/04

(52) U.S. Cl. CPC C07D 401/04; A01P 7/04

(57) ABSTRACT

The present disclosure is directed to simple, efficient and economically viable process for preparing anthranilamide compounds of Formula (I) useful as insecticidal agents for combating harmful pests, such as insects which destroy agricultural plants and crops wherein R is Cl or CN

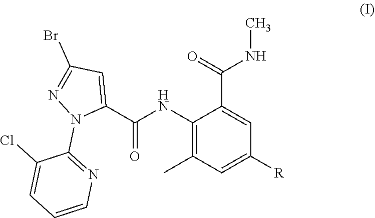

### FIELD OF THE INVENTION

The present disclosure pertains to technical field of organic synthesis. In particular, the present disclosure pertains to preparation of anthranilamide compounds useful as insecticidal agents.

### BACKGROUND OF THE INVENTION

Anthranilamide compounds are nitrogen-containing aromatic compounds that are selective targets of the ryanodine receptor in insects. Ryanodine receptors are calcium ion channels used in muscle function. When anthranilamides bind to insect ryanodine receptors, the insect's muscles contract, leading to paralysis and ultimately death for pests. The control of invertebrate pests is extremely important in achieving high crop efficiency. Damage by invertebrate pests to growing and stored agronomic crops can cause significant reduction in productivity and thereby result in increased costs to consumer.

Chlorantraniliprole, chemically known as 3-bromo-N-[4-chloro-2-methyl-6-(methylcarbamoyl)phenyl]-1-(3-chloropyridin-2-yl)-1H-pyrazole-5-carboxamide, is an anthranilamide insecticide. The mode of action of chlorantraniliprole is well known. It binds and activates ryanodine receptors (RyRs) located in the sarcoplasmic reticulum, to release stored intracellular calcium into cytoplasm. It stimulates the release and depletion of intracellular calcium stores from the sarcoplasmic reticulum of muscle cells, causing impaired muscle regulation, paralysis and ultimately death of sensitive species. This novel and unique calcium-induced muscle-contraction mode of action has been shown to be highly specific to insect ryanodine receptors.

Cyantraniliprole, chemically known as 3-Bromo-1-(3-chloro-2-pyridinyl)-N-[4-cyano-2-methyl-6-[(methylamino)carbonyl]phenyl]-1H-pyrazole-5-carboxamide, is an anthranilamide insecticide with a mode of action (ryanodine receptor activation) similar to chlorantraniliprole. It has root systemic activity with some translaminar movement and is effective against the larval stages of lepidopteran insects; and also on thrips, aphids, and some other chewing and sucking insects.

The PCT Publication No. WO02003/015519 (the WO'519 application) discloses chlorantraniliprole and processes for preparing chlorantraniliprole via a benzoxazinone intermediate. In the WO'519 application, the preparation of chlorantraniliprole is achieved by a two-step method, i.e., the first step is synthesis and isolation of a benzoxazinone intermediate, and the second step is conversion of the isolated benzoxazinone intermediate into the final product chlorantraniliprole. The synthetic routes disclosed in the WO'519 application for the preparation of chlorantraniliprole are depicted below:

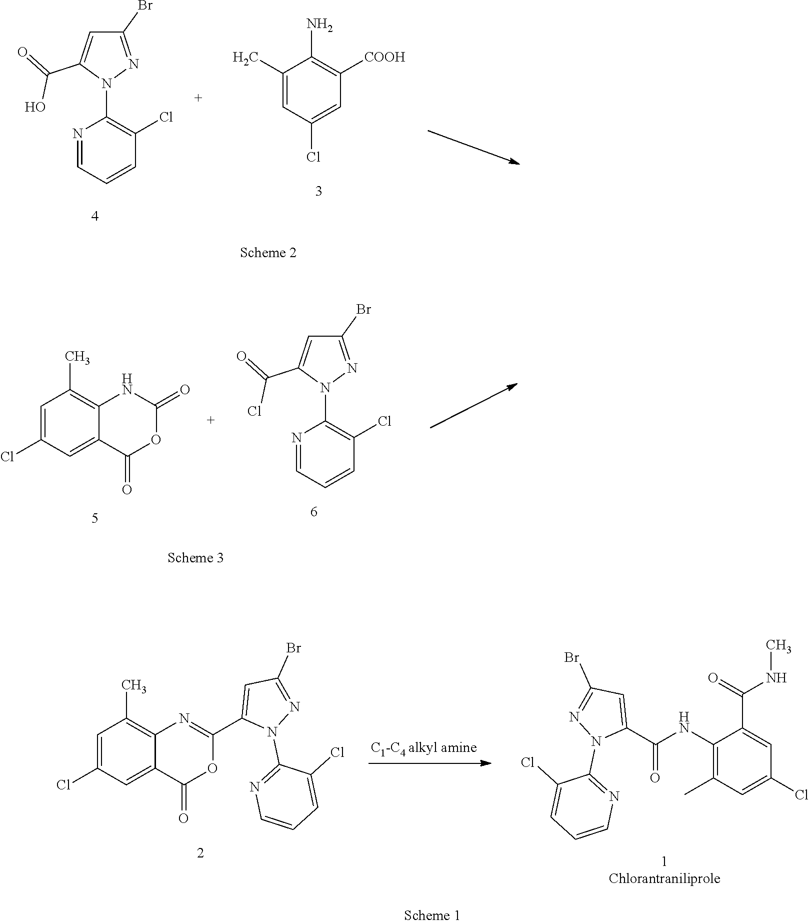 [SMILES: O=C(O)c1cc(Br)nn1-c1ncccc1Cl ; Cc1cc(Cl)cc(C(=O)O)c1N ; Cc1cc(Cl)cc2c(=O)oc(=O)[nH]c12 ; O=C(Cl)c1cc(Br)nn1-c1ncccc1Cl ; Cc1cc(Cl)cc2c(=O)oc(-c3cc(Br)nn3-c3ncccc3Cl)nc12 ; CNC(=O)c1cc(Cl)cc(C)c1NC(=O)c1cc(Br)nn1-c1ncccc1Cl]

In Scheme 2 of the WO'519 application, a benzoxazinone intermediate of Formula 2 is prepared via coupling of a pyrazolecarboxylic acid of Formula 4 with an anthranilic acid of Formula 3. The method of Scheme 2 involves sequential addition of methanesulfonyl chloride in the presence of a tertiary amine such as triethylamine or pyridine to a pyrazolecarboxylic acid of Formula 4, followed by the addition of an anthranilic acid of Formula 3, followed by a second addition of tertiary amine and methanesulfonyl chloride. Scheme 3 of the WO'519 application depicts an alternate preparation for benzoxazinone intermediate of Formula 2 involving coupling of a pyrazole acid chloride of Formula 6 with an isatoic anhydride of Formula 5 in pyridine or pyridine/acetonitrile to provide the benzoxazinone intermediate of Formula 2.

The PCT Publication No. WO02006/062978 discloses preparation of chlorantraniliprole by coupling a carboxylic acid of Formula 2 with an aniline of Formula 3 using a sulfonyl chloride, typically in the presence of a base and a solvent.

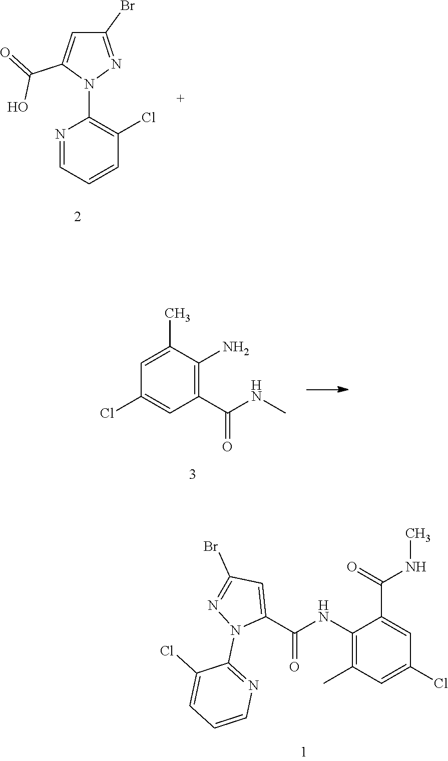 [SMILES: O=C(O)c1cc(Br)nn1-c1ncccc1Cl ; CNC(=O)c1cc(Cl)cc(C)c1N ; CNC(=O)c1cc(Cl)cc(C)c1NC(=O)c1cc(Br)nn1-c1ncccc1Cl]

The U.S. patent Publication No. 2010/0317864 discloses a method for preparation of chlorantraniliprole and cyantraniliprole. In particular, the following procedure to synthesize chlorantraniliprole or cyantraniliprole is disclosed:

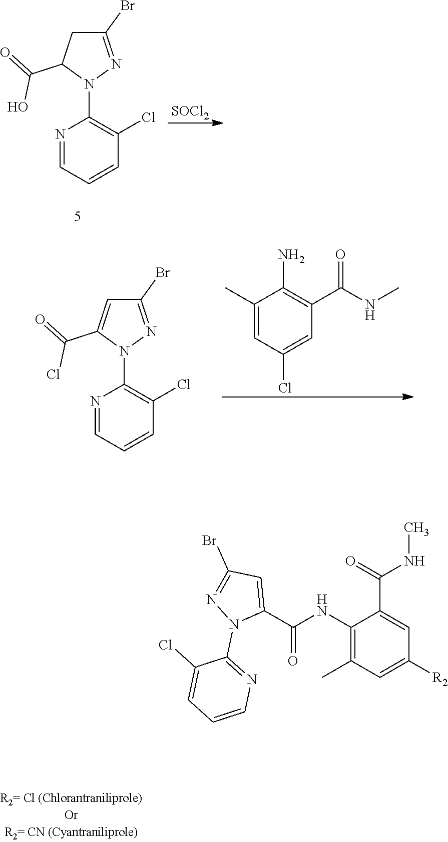

The PCT Publication No. WO02019/207595 describes a process for the preparation of chlorantraniliprole and cyantraniliprole, which comprises the steps of: a) reacting 3-bromo-1-(3-chloro-2-pyridyl)-1H-pyrazole-5-carboxylic acid with a chlorinating agent in a suitable solvent to obtain 3-bromo-1-(3-chloro-2-pyridyl)-1H-pyrazole-5-carboxylic acid chloride, and (b) reacting the 3-bromo-1-(3-chloro-2-pyridyl)-1H-pyrazole-5-carboxylic acid chloride in situ with a substituted aminobenzamide in presence of a base in a suitable solvent to obtain chlorantraniliprole and cyantraniliprole. The reaction scheme of this process is as follows:

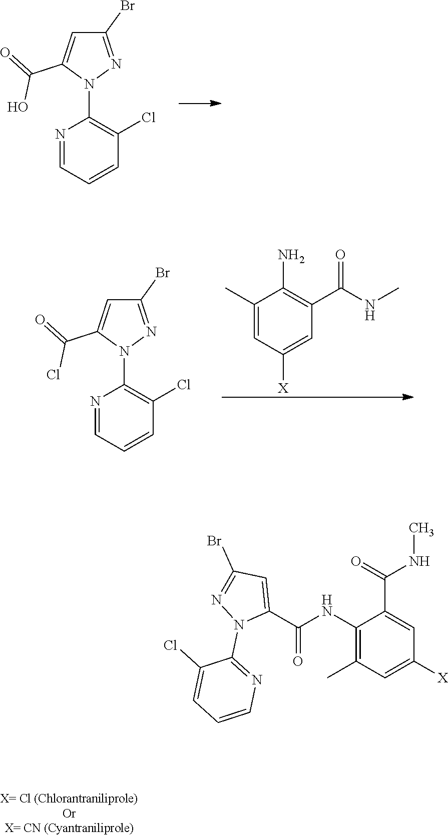

The PCT Publication No. WO02020/136480 (the WO'480 application) discloses a process wherein chlorantraniliprole and cyantraniliprole are prepared according to the method described in the U.S. Pat. Nos. 7,232,836 and 7,247,647, respectively. The crude compound thus obtained is then purified by treatment with water to obtain chlorantraniliprole or cyantraniliprole with a purity of about 97%. The process disclosed in the WO'480 application may be summarized as follows:

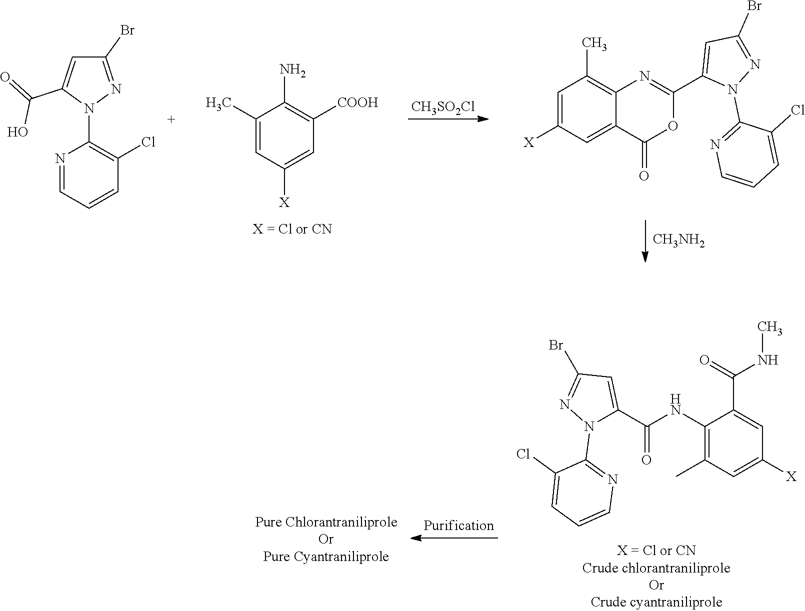

However, the above-mentioned prior art processes suffer distinct disadvantages which render them technically and economically objectionable. For example, the processes known in the art for preparing chlorantraniliprole or cyantraniliprole involve a greater number of individual and difficult steps, which include isolation of intermediate as well as purification of final product and this results in excessive production time and high costs, which in turn render the processes economically undesirable and less eco-friendly. Thus, the prior art processes are not suitable for commercial scale up.

Therefore, a need still exists in the art to provide a new and improved process for preparation of anthranilamide compounds such as chlorantraniliprole and cyantraniliprole. The present disclosure satisfies the existing needs, as well as others, and generally overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### OBJECTS OF THE INVENTION

It is an object of the present disclosure to provide a new and improved process for the preparation of anthranilamide compounds, which overcomes one or more disadvantages of the prior art specified above.

It is another object of the present disclosure to provide a process for the production of anthranilamide compounds in good yields and high purity characterized by simplicity and ease of operation.

It is yet another object of the present disclosure to provide an economical and eco-friendly process for the preparation of anthranilamide compounds such as chlorantraniliprole and cyantraniliprole.

### SUMMARY OF THE INVENTION

The foregoing and other objects are attained by the present disclosure, which in one aspect provides a single step process for preparation of an anthranilamide compound of Formula I in high purity and yield,

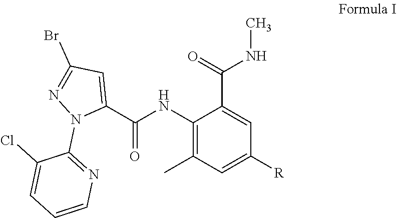

wherein R is Cl or CN

which comprises: forming a benzoxazinone of Formula II by reacting a pyrazolecarboxylic acid of Formula III, an anthranilic acid of Formula IV and a sulfonyl chloride in presence of a base selected from an inorganic or an organic base in a suitable solvent; and

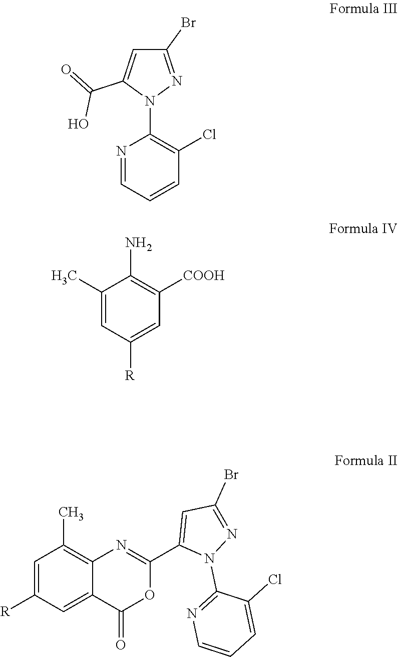

wherein R is as defined above,

converting the benzoxazinone of Formula II to the compound of Formula I, wherein the benzoxazinone of Formula II is not isolated after its formation and is in situ reacted with methylamine to yield the compound of Formula I.

In a second aspect, the present disclosure provides a simplified process for preparing anthranilamide compounds of Formula I in high purity and yield,

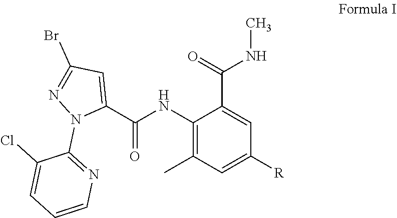

wherein R is Cl or CN

which comprises:
combining a pyrazolecarboxylic acid of Formula III, an anthranilic acid of Formula IV, a sulfonyl chloride and a base in a suitable solvent to form a reaction mixture;

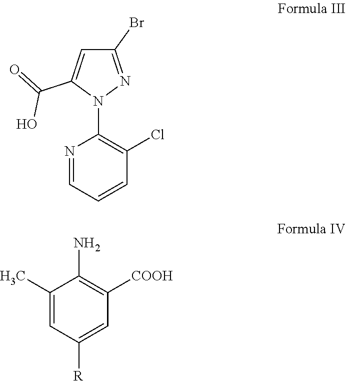

wherein R is as defined above

allowing the reaction mixture to react under conditions which result in the formation of a benzoxazinone of Formula II;

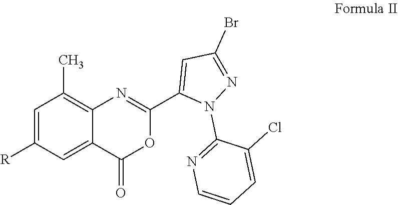

wherein R is as defined above

filtering the reaction mixture to yield a wet filter-cake comprising the benzoxazinone of Formula II; and
telescoping the wet filter-cake comprising the benzoxazinone of Formula II with methylamine to produce the compound of Formula I.

In a third aspect, the present disclosure provides a process for preparing a benzoxazinone of Formula II by reacting a pyrazolecarboxylic acid of Formula III, an anthranilic acid of Formula IV and a sulfonyl chloride in the presence of an inorganic base or an organic base, wherein the inorganic base is selected from sodium bicarbonate, sodium carbonate, potassium bicarbonate and potassium carbonate, and wherein the organic base is selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), triethylamine-DMAP, DABCO, N,N-dimethylaniline, and 4-(N,N-dimethyl)pyridine.

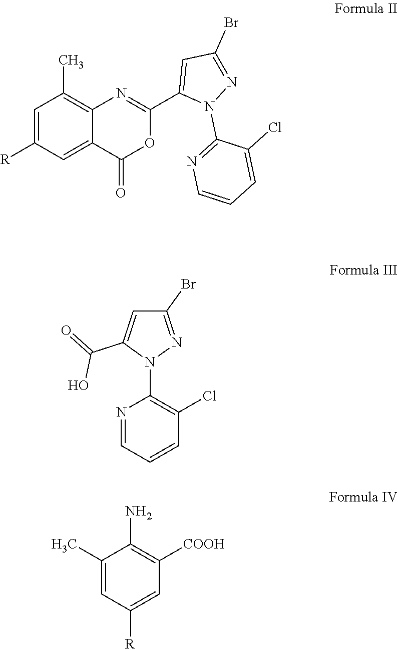

wherein R is Cl or CN

Various objects, features, aspects and advantages of the inventive subject matter will become more apparent from the following detailed description of preferred embodiments.

### DETAILED DESCRIPTION OF THE INVENTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, process conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

Aspects of the present disclosure relate to process for preparation of anthranilamide compounds of Formula I,

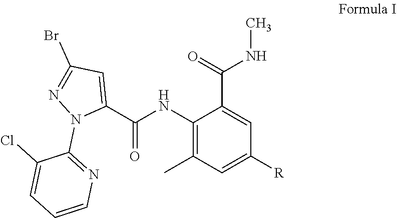

wherein R is Cl or CN

useful as insecticidal agents for combating harmful pests, such as insects which destroy agricultural plants and crops. The process disclosed herein enables to produce anthranilamide compounds of Formula I in a simple and easy manner as compared to known processes. Further, the process of the present disclosure affords the compound of Formula I in good yield and in substantially pure form, thus generating economic benefits and obviating the necessity of subjecting it to further purification. Also, the process disclosed herein is environment friendly and easy to scale up for industrial manufacture with improved quality of anthranilamide compounds such as chlorantraniliprole and cyantraniliprole.

In one embodiment, the compound of Formula IA is chlorantraniliprole having the Formula (IA):

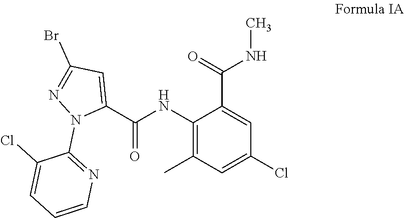 [SMILES: CNC(=O)c1cc(Cl)cc(C)c1NC(=O)c1cc(Br)nn1-c1ncccc1Cl]

In another embodiment, the compound of Formula IB is cyantraniliprole having the Formula (IB):

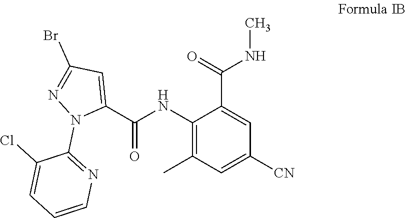 [SMILES: CNC(=O)c1cc(C#N)cc(C)c1NC(=O)c1cc(Br)nn1-c1ncccc1Cl]

In a first aspect, the present disclosure provides a single step process for preparation of an anthranilamide compound of Formula I,

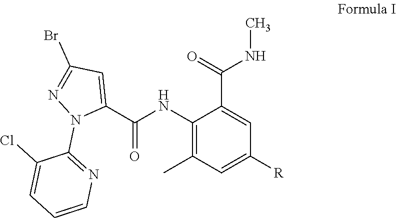

wherein R is Cl or CN

which comprises: forming a benzoxazinone of Formula II by reacting a pyrazolecarboxylic acid of Formula III, an anthranilic acid of Formula IV and a sulfonyl chloride in presence of a base in a suitable solvent; and

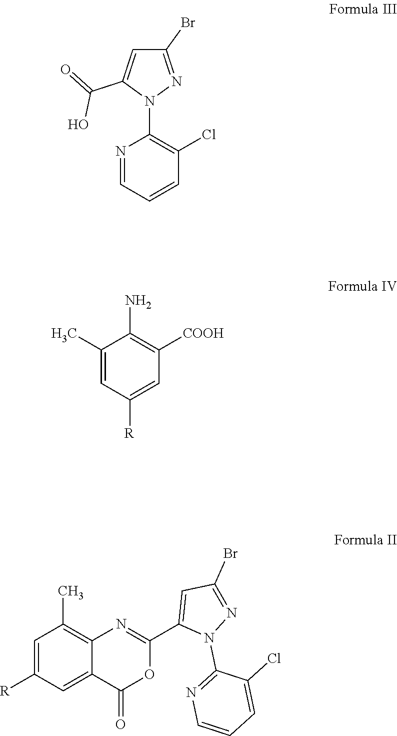

wherein R is as defined above,

converting the benzoxazinone of Formula II to the compound of Formula I, wherein the benzoxazinone of Formula II is not isolated after its formation and is in situ reacted with methylamine in the same reaction mixture to yield the compound of Formula I. The process is therefore much simpler than the processes of prior art, first because it obviates isolation and/or purification of the benzoxazinone intermediate of Formula II. Further, the process according to the first aspect of the present disclosure affords the compound of Formula I in substantially pure form, thus obviating the necessity of subjecting it to further purification.

In various embodiments, the process according to the first aspect of the present disclosure produces the anthranilamide compound of Formula I in a purity of at least about 98%, as measured by HPLC.

In various embodiments, the sulfonyl chloride used in the process according to the first aspect of the present disclosure is selected from methanesulfonyl chloride, benzenesulfonyl chloride and p-toluenesulfonyl chloride.

The base used in the process according to the first aspect of the present disclosure can be either an organic or inorganic base. A preferred organic base for use herein is selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), DABCO, N,N-dimethylaniline, and 4-(N,N-dimethyl)pyridine. A preferred inorganic base is selected from the group consisting of sodium bicarbonate, sodium carbonate, potassium bicarbonate and potassium carbonate. A more preferred base for use herein is 3-picoline, N-methylimidazole, 4-dimethylaminopyridine (DMAP), triethylamine-DMAP, sodium acetate or potassium carbonate.

While any suitable solvent may be employed in the process according to the first aspect of the present disclosure, preferably, the solvent is selected from the group consisting of acetonitrile, dichloroethane, dichloromethane, toluene, chlorobenzene, dichlorobenzene, acetone, diethyl ketone, iso-butyl methyl ketone, methyl acetate, ethyl acetate, n-propyl acetate, iso-propyl acetate, N,N-dimethyl formamide, N,N-dimethyl acetamide, N-methyl-2-pyrrolidone, dimethyl sulfoxide, tetrahydrofuran, 2-methyltetrahydrofuran, tert-butanol and mixtures of two or more of the above. A more preferred solvent for use herein is selected from the group consisting of acetonitrile, dichloroethane, dichloromethane, toluene, acetone, ethyl acetate, iso-propyl acetate, tetrahydrofuran, 2-methyltetrahydrofuran, and mixtures thereof.

In at least one embodiment of the first aspect, a benzoxazinone of Formula II is formed by combining a sulfonyl chloride with a mixture comprising a pyrazolecarboxylic acid of Formula III, an anthranilic acid of Formula IV, a base and a solvent, and then maintaining the resultant reaction mixture at a temperature ranging from about −5° C. to about 20° C. for a period of time sufficient for the reaction to go to completion.

In various embodiments of the first aspect, the compound of Formula I is obtained by contacting methylamine with the in-situ formed benzoxazinone of Formula II. The reaction with the in-situ formed benzoxazinone of Formula II may conveniently be effected at any suitable temperature, for example from room temperature to reflux temperature of the solvent. In one embodiment, the in-situ formed benzoxazinone of Formula II is treated with methylamine at a temperature of from about 30° C. to 80° C. to obtain the compound of Formula I. The methylamine for use herein can be aqueous methylamine or alcoholic methylamine, such as methanolic methylamine.

In at least one embodiment, the process according to the first aspect of the present disclosure provides the anthranilamide compound of Formula I at a yield of greater than about 85% having a purity of at least about 98%, as measured by HPLC.

In a second aspect, there is provided a simplified process for preparing anthranilamide compounds of Formula I in high purity and yield,

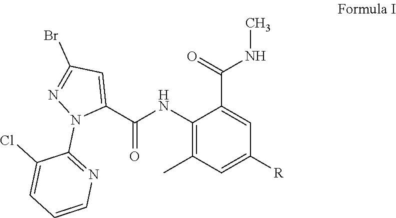

wherein R is Cl or CN

which comprises:
combining a pyrazolecarboxylic acid of Formula III, an anthranilic acid of Formula IV, a sulfonyl chloride and a base in a suitable solvent to form a reaction mixture;

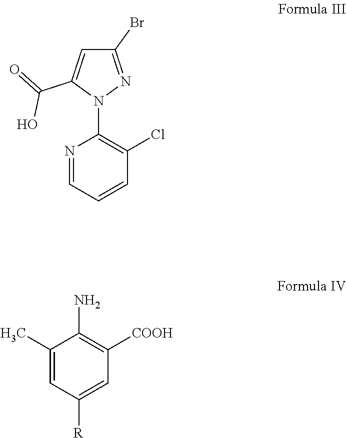

wherein R is as defined above
allowing the reaction mixture to react under conditions which result in the formation of a benzoxazinone of Formula II;

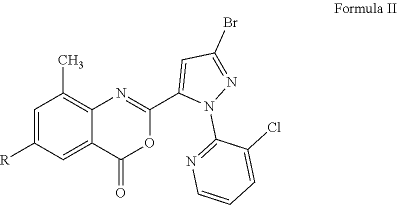

wherein R is as defined above
filtering the reaction mixture to yield a wet filter-cake comprising the benzoxazinone of Formula II; and
telescoping the wet filter-cake comprising the benzoxazinone of Formula II with methylamine to produce the compound of Formula I.

The process according to the second aspect of the present disclosure is simplified with respect to processes of prior art because, unlike processes of prior art, in the process according to the second aspect of the present disclosure, the crude wet benzoxazinone filter-cake obtained from the reaction mixture does not need to be purified and/or dried before being treated with methylamine to convert it to anthranilamide compound of Formula I. Thus, the process according to the second aspect of the present disclosure is particularly advantageous in that it makes it possible to produce highly pure anthranilamide compound of Formula I in a simple and cost-effective manner, which was not the case of the processes of prior art.

In various embodiments, the process according to the second aspect of the present disclosure produces the anthranilamide compound of Formula I at a yield of greater than about 85% having a purity of at least about 98%, as measured by HPLC.

In certain embodiments, the process according to the second aspect of the present disclosure provides the anthranilamide compound of Formula I having a purity of at least about 99%, as measured by HPLC.

In various embodiments, the sulfonyl chloride used in the process according to the second aspect of the present disclosure can be selected from methanesulfonyl chloride, benzenesulfonyl chloride and p-toluenesulfonyl chloride.

The base used in the process according to the second aspect of the present disclosure can be either an organic or inorganic base. A preferred organic base for use herein is selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), DABCO, N,N-dimethylaniline, and 4-(N,N-dimethyl)pyridine. A preferred inorganic base is selected from the group consisting of sodium bicarbonate, sodium carbonate, potassium bicarbonate and potassium carbonate. A more preferred base for use herein is 3-picoline, N-methylimidazole, 4-dimethylaminopyridine (DMAP), triethylamine-DMAP, sodium acetate or potassium carbonate.

The solvent which is used in the process according to the second aspect of the present disclosure may be any solvent suitable for forming the benzoxazinone of Formula II. Examples of suitable solvent include, but not limited to, acetonitrile, dichloroethane, dichloromethane, toluene, chlorobenzene, dichlorobenzene, acetone, diethyl ketone, iso-butyl methyl ketone, methyl acetate, ethyl acetate, n-propyl acetate, iso-propyl acetate, N,N-dimethyl formamide, N,N-dimethyl acetamide, N-methyl-2-pyrrolidone, dimethyl sulfoxide, tetrahydrofuran, 2-methyltetrahydrofuran, tert-butanol and mixtures of two or more of the above. A more preferred solvent for use herein is selected from the group consisting of acetonitrile, dichloroethane, dichloromethane, toluene, acetone, ethyl acetate, iso-propyl acetate, tetrahydrofuran, 2-methyltetrahydrofuran, and mixtures thereof.

In certain embodiments of the second aspect, a benzoxazinone of Formula II is formed by combining a sulfonyl chloride with a mixture of a pyrazolecarboxylic acid of Formula III, an anthranilic acid of Formula IV, a base and a solvent, and then maintaining the resultant reaction mixture at a temperature ranging from about −5° C. to about 20° C. for a period of time sufficient for the reaction to go to completion.

In various embodiments of the second aspect, the compound of Formula I is obtained by contacting methylamine with a wet filter-cake comprising the benzoxazinone of Formula II. In one preferred embodiment, the compound of Formula I is produced by treating a wet filter-cake of benzoxazinone of Formula II with methylamine at a temperature ranging from about 30° C. to 80° C. The methylamine for use herein can be aqueous methylamine or alcoholic methylamine, such as methanolic methylamine.

In a third aspect, there is provided a process for preparing a benzoxazinone of Formula II by reacting a pyrazolecarboxylic acid of Formula III, an anthranilic acid of Formula IV and a sulfonyl chloride in the presence of an inorganic base or an organic base, wherein the inorganic base is selected from sodium bicarbonate, sodium carbonate, potassium bicarbonate and potassium carbonate, and wherein the organic base is selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), triethylamine-DMAP, DABCO, N,N-dimethylaniline, and 4-(N,N-dimethyl)pyridine.

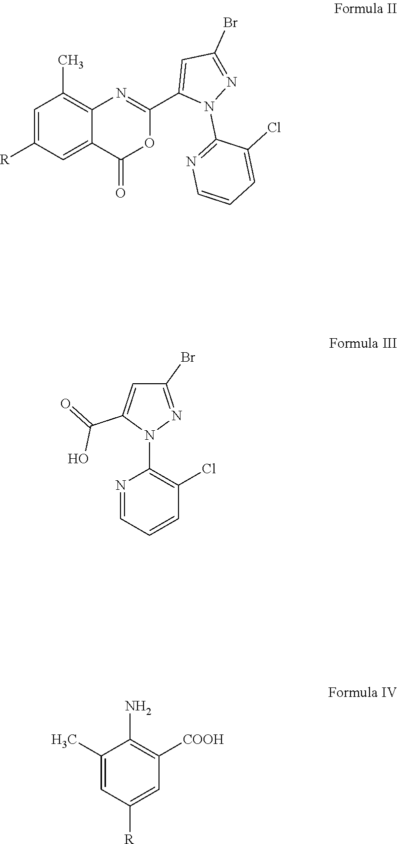

wherein R is Cl or CN

In various embodiments, the sulfonyl chloride used in the process according to the third aspect of the present disclosure is selected from methanesulfonyl chloride, benzenesulfonyl chloride and p-toluenesulfonyl chloride.

In various embodiments, the base used in the process according to the third aspect of the present disclosure is preferably selected from 3-picoline, N-methylimidazole, 4-dimethylaminopyridine (DMAP), triethylamine-DMAP, sodium acetate and potassium carbonate.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example 1: Preparation of Chlorantraniliprole

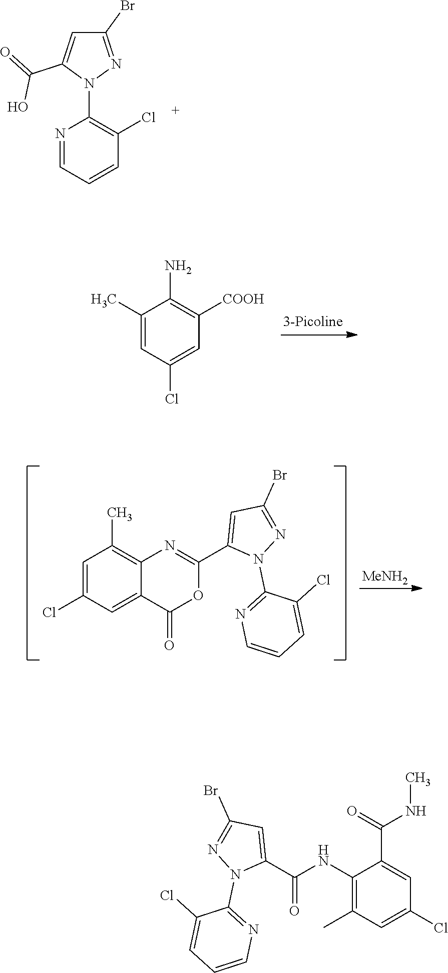

To a solution of 2-amino-5-chloro-3-methylbenzoic acid (4.4 g, 23.8 mmol), 3-bromo-1-(3-chloropyridin-2-yl)-1H-pyrazole-5-carboxylic acid (7.2 g, 23.8 mmol) and 3-Picoline (7.7 g, 82.7 mmol) in 35 ml of acetonitrile was slowly added methane sulfonyl chloride (8.2 g, 71.3 mmol) at 5 to −5° C. temperature. The progress of the reaction was monitored by HPLC. Upon completion of the reaction, aqueous methyl amine solution (11.1 g; 40% by wt.) was added at ambient temperature. The reaction mass was heated to 40-60° C. and stirred for 60 min at the same temperature. The consumption of in-situ formed benzoxazinone intermediate (2-(3-bromo-1-(3-chloropyridin-2-yl)-1H-pyrazol-5-yl)-6-chloro-8-methyl-4H-enzo [d][1,3] oxazin-4-one) was monitored by HPLC. Upon completion of the reaction, the reaction mass containing chlorantraniliprole was cooled to 5-10° C., stirred for about 60 min at the same temperature and filtered. The wet cake was washed with acetonitrile and dried to afford 9.89 g of chlorantraniliprole (Yield 86%; and Purity 98.5% as determined by HPLC analysis).

### Example 2: Preparation of Chlorantraniliprole

To a solution of 2-amino-5-chloro-3-methylbenzoic acid (3.9 g, 21.01 mmol), 3-bromo-1-(3-chloropyridin-2-yl)-1H-pyrazole-5-carboxylic acid (6.3 g, 20.8 mmol) and 3-Picoline (7.8 g, 83.7 mmol) in 30 ml of 2-methyl THF was slowly added methane sulfonyl chloride (8.3 g, 72.4 mmol) at 5 to −5° C. temperature. The progress of the reaction was monitored by HPLC. Upon completion of the reaction, aqueous methyl amine solution (9.8 g; 40% by wt.) was added at the ambient temperature. The reaction mass thereto was heated to 50-70° C. and stirred for 60 min at the same temperature. The consumption of in-situ formed benzoxazinone intermediate was monitored by HPLC. Upon completion of the reaction, the reaction mass mainly containing chlorantraniliprole was cooled to 5-10° C., stirred at for about 60 min at the same temperature and filtered. The wet cake was washed with acetonitrile to afford 8.6 g chlorantraniliprole in high yield and purity (Yield 86%; and Purity 98.3% as determined by HPLC analysis).

### Example 3: Preparation of Chlorantraniliprole

To a solution of 2-amino-5-chloro-3-methylbenzoic acid (3.8 g, 20.49 mmol), 3-bromo-1-(3-chloropyridin-2-yl)-1H-pyrazole-5-carboxylic acid (6.2 g, 20.49 mmol) and 3-Picoline (7.6 g, 81.6 mmol) in 35 ml of acetonitrile was slowly added methane sulfonyl chloride (8.1 g, 70.7 mmol) at 5 to −5° C. temperature. The progress of the reaction was monitored by HPLC. Upon completion of the reaction, methanolic methyl amine solution (9.5 g; 40% by wt.) was slowly added at the ambient temperature. The reaction mass thereto was heated to 35-55° C. and stirred for 50 min at the same temperature. The consumption of in-situ formed benzoxazinone intermediate was monitored by HPLC. Upon completion of the reaction, the reaction mass mainly containing chlorantraniliprole was cooled to 5-10° C., stirred at for about 60-70 min at the same temperature and filtered. The wet cake was washed with acetonitrile to afford 8.3 g chlorantraniliprole in high yield and purity (Yield 84%; and Purity 98.4% as determined by HPLC analysis).

### Example 4: Preparation of Chlorantraniliprole

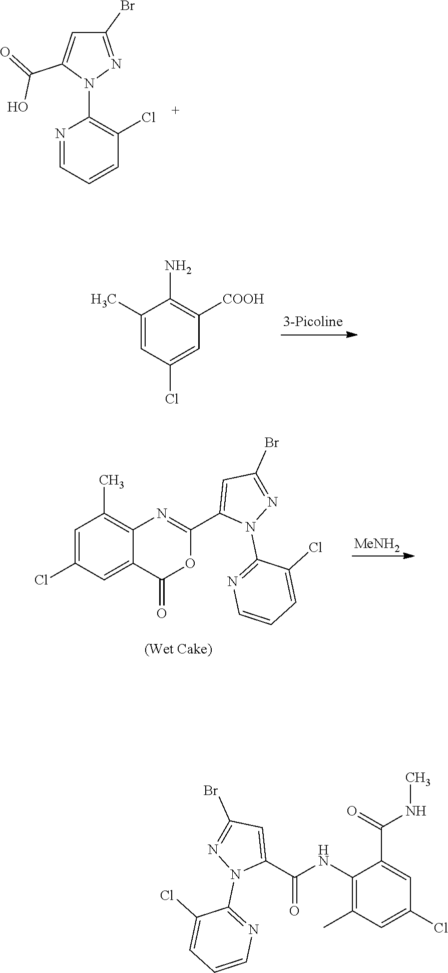

To a solution of 5-chloro-2-amino-3-methyl benzoic acid (4.32 g, 23.27 mmol), 3-bromo-1-(3-chloropyridin-2-yl)-1H-pyrazole-5-carboxylic acid (7.04 g, 23.27 mmol) and 3-Picoline (7.58 g, 81.44 mmol) in 15 ml acetonitrile, a solution of (13.04 g, 113.9 mmol) of methane sulfonyl chloride in 13.0 ml of acetonitrile was slowly added at 0-10° C. The reaction mass was maintained at the same temperature for about 60 min and progress of the reaction was monitored by HPLC. Upon completion of the reaction, the reaction mass was filtered to obtain the wet cake of benzoxazinone intermediate (2-(3-bromo-1-(3-chloropyridin-2-yl)-1H-pyrazol-5-yl)-6-chloro-8-methyl-4H-enzo[d][1,3]oxazin-4-one) (Purity by HPLC 99.5%) which was further telescoped to chlorantraniliprole preparation by suspending the wet cake of the benzoxazinone intermediate in acetonitrile (78 ml) and slow addition of aqueous methyl amine solution (3.9 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with acetonitrile and dried to afford 10 g of chlorantraniliprole in high purity (Yield 89%; Purity 99.5% as determined by HPLC analysis).

### Example 5: Preparation of Chlorantraniliprole

To a solution of 5-chloro-2-amino-3-methyl benzoic acid (0.61 g, 33.0 mmol), 3-bromo-1-(3-chloropyridin-2-yl)-1H-pyrazole-5-carboxylic acid (1.00 g, 33.0 mmol) and 3-Picoline (1.05 g, 112.0 mmol) in 15 ml acetonitrile, a solution of (1.88 g, 98.0 mmol) of p-toluene sulfonyl chloride in 5 ml of acetonitrile was slowly added at 0-10° C. The reaction mass was maintained at the same temperature for about 60 min and progress of the reaction was monitored by HPLC. Upon completion of the reaction, the reaction mass was filtered to obtain wet cake of benzoxazinone intermediate (2-(3-bromo-1-(3-chloropyridin-2-yl)-1H-pyrazol-5-yl)-6-chloro-8-methyl-4H-enzo[d][1,3]oxazin-4-one) (Purity by HPLC 98.8%) which was further telescoped to chlorantraniliprole preparation by suspending the wet cake of the benzoxazinone intermediate in acetonitrile (15 ml) and slow addition of aqueous methyl amine solution (0.38 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with cold acetonitrile and dried to afford 1.5 g of chlorantraniliprole in high purity (Yield 94%; Purity 98.65% as determined by HPLC analysis).

### Example 6: Preparation of Chlorantraniliprole

To a solution of 5-chloro-2-amino-3-methyl benzoic acid (4.43 g, 23.86 mmol), 3-bromo-1-(3-chloropyridin-2-yl)-1H-pyrazole-5-carboxylic acid (7.21 g, 23.86 mmol), triethylamine (7.30 g, 72.27 mmol) and DMAP (4.9 g, 40.44 mmol) in 15 ml of acetonitrile, a solution of methane sulfonyl chloride (8.30 g, 72.45 mmol) in 10.0 ml acetonitrile was slowly added at 0-10° C. The same temperature was maintained for about 60 min and the progress of the reaction was monitored by HPLC. Upon completion of the reaction, the reaction mass thereto was filtered to obtain wet cake of benzoxazinone intermediate, 2-(3-bromo-1-(3-chloropyridin-2-yl)-1H-pyrazol-5-yl)-6-chloro-8-methyl-4H-enzo[d][1,3 ]oxazin-4-one (Purity by HPLC: 98.9%), which was further telescoped to chlorantraniliprole preparation by suspending the wet cake of the benzoxazinone intermediate in acetonitrile (55 ml) and slow addition of aqueous methyl amine solution (3.91 g; 40% by wt.) at 20-30° C. Thereafter, the reaction mass was heated to 50-55° C. and stirred for 60 min at the same temperature. The progress of the reaction was monitored by TLC/HPLC. Upon completion of the reaction, the reaction mass was filtered, washed with cold acetonitrile and dried to afford 9.67 g of chlorantraniliprole in high purity (Yield 84%; Purity 99.1% as determined by HPLC analysis).

### Example 7: Preparation of Chlorantraniliprole

To a solution of 5-chloro-2-amino-3-methyl benzoic acid (4.25 g, 22.89 mmol), 3-bromo-1-(3-chloropyridin-2-yl)-1H-pyrazole-5-carboxylic acid (6.9 g, 22.88 mmol), and diisopropylethylamine (19.7 g, 152.41 mmol) in 15 ml acetonitrile, a solution of (12.5 g, 109.11 mmol) of methane sulfonyl chloride in 10.0 ml acetonitrile was slowly added at 5-15° C. The same temperature was maintained for about 60 min and the progress of the reaction was monitored by HPLC. Upon completion of the reaction, the reaction mass thereto was filtered to obtain wet cake of benzoxazinone intermediate, 2-(3-bromo-1-(3-chloropyridin-2-yl)-1H-pyrazol-5-yl)-6-chloro-8-methyl-4H-enzo [d][1,3 ]oxazin-4-one in high purity (99.0%), which was further telescoped to chlorantraniliprole preparation by suspending the wet cake of the benzoxazinone intermediate in acetonitrile (55 ml) and slow addition of aqueous methyl amine solution (3.91 g; 40% by wt.) at 20-30° C. Thereafter, the reaction mass was heated to 50-55° C. and stirred for 60 min at the same temperature. The progress of the reaction was monitored by TLC/HPLC. Upon completion of the reaction, the reaction mass was filtered, washed with cold acetonitrile and dried to afford 9.1 g of chlorantraniliprole in high purity (Yield 82.5%; Purity 99.1% as determined by HPLC analysis).

### Example 8: Preparation of Chlorantraniliprole

To a solution of 5-chloro-2-amino-3-methyl benzoic acid (3.05 g, 16.43 mmol), 3-bromo-1-(3-chloropyridin-2-yl)-1H-pyrazole-5-carboxylic acid (5.0 g, 16.53 mmol) and 3-Picoline (10.46 g, 112.32 mmol) in 20 ml acetone, a solution of methane sulfonyl chloride (9.08 g, 79.30 mmol) in 5.0 ml acetone was slowly added at 5-10° C. The same temperature was maintained for about 60 min and the progress of the reaction was monitored by TLC. Upon completion of the reaction, the reaction mass thereto was filtered to obtain wet cake of benzoxazinone intermediate, 2-(3-bromo-1-(3-chloropyridin-2-yl)-1H-pyraz 01-5-yl)-6-chloro-8-methyl-4H-enzo[d][1,3]oxazin-4-one in high purity (99.27%), which was further telescoped to chlorantraniliprole preparation by suspending the wet cake of the benzoxazinone intermediate in acetone (25 ml) and slow addition of aqueous methyl amine solution (2.56 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with cold acetone and dried to afford 7.27 g of chlorantraniliprole in high purity (Yield 91%; Purity 99.12% as determined by HPLC analysis).

### Example 9: Preparation of Chlorantraniliprole

To a solution of 5-chloro-2-amino-3-methyl benzoic acid (3.05 g, 16.43 mmol), 3-bromo-1-(3-chloropyridin-2-yl)-1H-pyrazole-5-carboxylic acid (5.00 g, 16.53 mmol) and 3-Picolin (7.32 g, 78.62 mmol) in 25 ml of 2-methyl THF, methane sulfonyl chloride (6.36 g, 55.51 mmol) was slowly added at 5-10° C. The same temperature was maintained for about 60 min and the progress of the reaction was monitored by TLC. Upon completion of the reaction, the reaction mass thereto was filtered to obtain wet cake of benzoxazinone intermediate, 2-(3-bromo-1-(3-chloropyridin-2-yl)-1H-pyrazol-5-yl)-6-chloro-8-methyl -4H-enzo [d][1,3 ]oxazin-4-one (Chromatographic purity by HPLC: 97.64%) which was further telescoped to chlorantraniliprole preparation by suspending the wet cake of the benzoxazinone intermediate in 1,2-dichloroethane (25 ml) and slow addition of aqueous methyl amine solution (2.15 g; 40% by wt.) at 20-30° C. Thereafter, the reaction mass was heated to 40-45° C. and stirred for 60 min at the same temperature. The progress of the reaction was monitored by TLC/HPLC. Upon completion of the reaction, the reaction mass was filtered, washed with cold 1,2-dichloraethane and dried to afford 7.03 g of chlorantraniliprole in high purity (Yield 88%; Purity 99.28% as determined by HPLC analysis).

### Example 10: Preparation of Chlorantraniliprole

To a solution of 5-chloro-2-amino-3-methyl benzoic acid (2.44 g, 13.13 mmol), 3-bromo-1-(3-chloropyridin-2-yl)-1H-pyrazole-5-carboxylic acid (3.97 g, 13.13 mmol) and potassium carbonate (5.51 g, 39.90 mmol) in 15 ml of acetonitrile, a solution of methane sulfonyl chloride (4.57 g, 39.85 mmol) in 10 ml of acetonitrile was slowly added at 5-15° C. The same temperature was maintained for about 60 min and the progress of the reaction was monitored by HPLC. Upon completion of the reaction, the reaction mass thereto was filtered followed by water wash to obtain wet cake of benzoxazinone intermediate, 2-(3-bromo-1-(3-chloropyridin-2-yl)-1H-pyrazol-5-yl)-6-chloro-8-methyl-4H-enzo[d][1,3 ]oxazin-4-one in high purity (98.2%), which was further telescoped to chlorantraniliprole preparation by suspending the wet cake of the benzoxazinone intermediate in acetonitrile (35 ml) and slow addition of aqueous methyl amine solution (2.15 g; 40% by wt.) at 20-30° C. Thereafter, the reaction mass was heated to 45-55° C. and stirred for 60 min at the same temperature. The progress of the reaction was monitored by TLC/HPLC. Upon completion of the reaction, the reaction mass was filtered, washed with cold acetonitrile and dried to afford 5.58 g of chlorantraniliprole in high purity (Yield 88%; Purity 98.9% as determined by HPLC analysis).

### Example 11: Preparation of Chlorantraniliprole

To a solution of 5-chloro-2-amino-3-methyl benzoic acid (1.55 g, 8.35 mmol), 3-bromo-1-(3-chloropyridin-2-yl)-1H-pyrazole-5-carboxylic acid (2.53 g, 8.36 mmol) and 3-Picolin (2.73 g, 29.31 mmol) in 15 ml acetonitrile, a solution of methane sulfonyl chloride (2.91 g, 25.36 mmol) of in 7 ml acetonitrile was slowly added at 0-10° C. The same temperature was maintained for about 60 min and the progress of the reaction was monitored by HPLC. Upon completion of the reaction, the reaction mass was filtered to obtain wet cake of benzoxazinone intermediate in high purity (99.2%), which was further telescoped to chlorantraniliprole preparation by suspending the wet cake of the benzoxazinone intermediate in 1,2-dichloroethane (12 ml) and slow addition of aqueous methyl amine solution (1.37 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with cold 1,2-dichloroethane and dried to afford 3.64 g of chlorantraniliprole in high purity (Yield 90%; Purity 99.1% as determined by HPLC analysis).

### Example 12: Preparation of Chlorantraniliprole

To a solution of 5-chloro-2-amino-3-methyl benzoic acid (5.32 g, 28.64 mmol), 3-bromo-1-(3-chloropyridin-2-yl)-1H-pyrazole-5-carboxylic acid (8.66 g, 28.64 mmol) and 3-Picolin (9.36 g, 100.50 mmol) in 30 ml acetonitrile, a solution of methane sulfonyl chloride (9.96 g, 86.94 mmol of in 15 ml acetonitrile was slowly added at 0-10° C. The same temperature was maintained for about 60 min and the progress of the reaction was monitored by TLC. Upon completion of the reaction, the reaction mass was filtered to obtain wet cake of benzoxazinone intermediate in high purity (99.4%), which was further telescoped to chlorantraniliprole preparation by suspending the wet cake of the benzoxazinone intermediate in acetonitrile (75 ml) and slow addition of methanolic methyl amine solution (5.40 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with cold acetonitrile and dried to afford 11.6 g of chlorantraniliprole in high purity (Yield 83.84%; Purity 99.4% as determined by HPLC analysis).

### Example 13: Preparation of Chlorantraniliprole

To a solution of 5-chloro-2-amino-3-methyl benzoic acid (3.05 g, 16.43 mmol), 3-bromo-1-(3-chloropyridin-2-yl)-1H-pyrazole-5-carboxylic acid (5.00 g, 16.53 mmol) and Sodium acetate trihydrate (12 g, 88.18 mmol) in 25 ml acetonitrile, methane sulfonyl chloride in (6.36 g, 55.51 mmol) was slowly added at 0-10° C. The reaction mass was maintained at the same temperature for about 60 min and progress of the reaction was monitored by TLC. Upon completion of the reaction, the reaction mass was filtered to obtain wet cake of benzoxazinone intermediate (2-(3-bromo-1-(3-chloropyridin-2-yl)-1H-pyrazol-5-yl)-6-chloro-8-methyl-4H-enzo[d][1,3]oxazin-4-one) (Purity by HPLC 98.8%) which was further telescoped to chlorantraniliprole preparation by suspending the wet cake of the benzoxazinone intermediate in 25 ml acetonitrile and slow addition of aqueous methyl amine solution (2.56 g; 40% by wt.) at 20-30° C. Thereafter, the reaction mass was heated to 50-60° C. and stirred for 60 min at the same temperature. The progress of the reaction was monitored by TLC/HPLC. Upon completion of the reaction, the reaction mass was filtered, washed with cold acetonitrile and dried to afford 6.9 g of chlorantraniliprole in high purity (Yield 86.4%; Purity 99.10% as determined by HPLC analysis).

### Example 14: Preparation of Cyantraniliprole

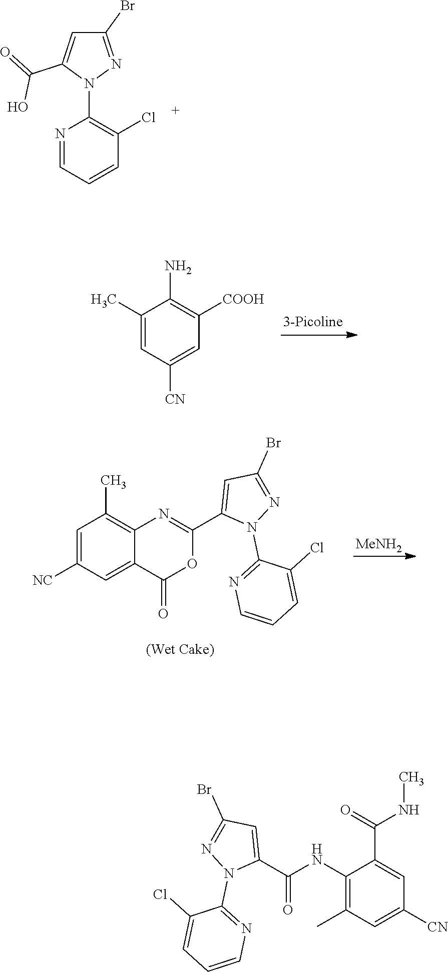

To a solution of 5-cyano-2-amino-3-methyl benzoic acid (3.50 g, 19.87 mmol), 3-bromo-1-(3-chloropyridin-2-yl)-1H-pyrazole-5-carboxylic acid (6.02 g, 19.90 mmol) and 3-Picoline (6.56 g, 70.41 mmol) in 20 ml acetonitrile, a solution of methane sulfonyl chloride (6.95 g, 60.68 mmol) in 10.0 ml acetonitrile was slowly added at 0-15° C. The same temperature was maintained for about 60 min and the progress of the reaction was monitored by HPLC. Upon completion of the reaction, the reaction mass was filtered to obtain wet cake of cyano benzoxazinone intermediate (2-(3-bromo-1-(3-chloropyridin-2-yl)-1H-pyrazol-5-yl)-8-methyl-4-oxo-4H-benzo[d][1,3]oxazine-6-carbonitrile) (Purity =99.2%), which was further telescoped to Cyantraniliprole preparation by suspending the wet cake of the cyano benzoxazinone intermediate in acetonitrile (30 ml) and slow addition of aqueous methyl amine solution (3.09 g; 40% by wt.) at 25-30° C. Thereafter, the reaction mass was heated to 45-55° C. and stirred for 60 min at the same temperature. The progress of the reaction was monitored by TLC/HPLC. Upon completion of the reaction, the reaction mass was filtered, washed with cold acetonitrile and dried to afford 8.56 g of Cyantraniliprole in high purity (Yield 89%; Purity 98.9% as determined by HPLC analysis).

1. A process for preparation of a compound of Formula I 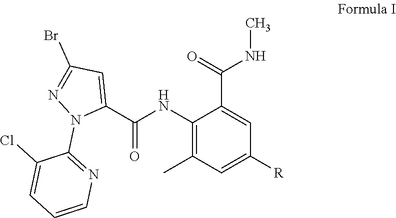
wherein R is Cl or CN
which comprises: forming a benzoxazinone of Formula II by reacting a pyrazolecarboxylic acid of Formula III, an anthranilic acid of Formula IV and a sulfonyl chloride in presence of a base in a solvent; and 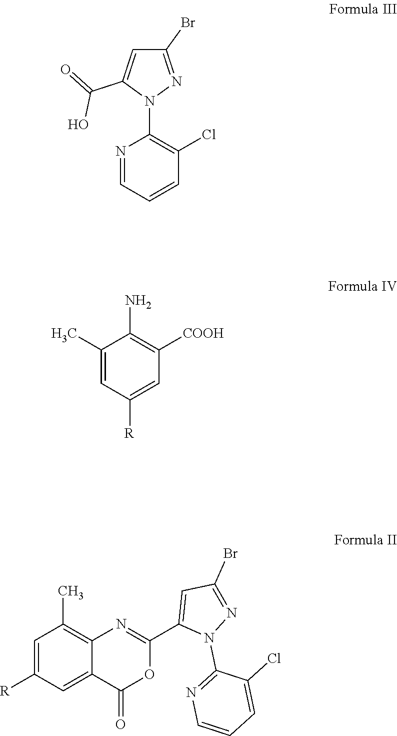
wherein R is Cl or CN
converting the benzoxazinone of Formula II, wherein the benzoxazinone of Formula II is not isolated after its formation, to the compound of Formula I.

2. The process as claimed in claim 1, wherein the sulfonyl chloride is methanesulfonyl chloride, benzenesulfonyl chloride or p-toluenesulfonyl chloride.

3. The process as claimed in claim 1, wherein the base is an inorganic or organic base.

4. The process as claimed in claim 3, wherein the base is an inorganic base selected from the group consisting of sodium bicarbonate, sodium carbonate, potassium bicarbonate and potassium carbonate.

5. The process as claimed in claim 3, wherein the base is an organic base selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), triethylamine-DMAP, DABCO, N,N-dimethylaniline, and 4-(N,N-dimethyl)pyridine.

6. The process as claimed in claim 3, wherein the base is selected from the group consisting of 3-picoline, N-methylimidazole, 4-dimethylaminopyridine (DMAP), triethylamine-DMAP, sodium acetate and potassium carbonate.

7. The process as claimed in claim 1, wherein the solvent is selected from the group consisting of acetonitrile, dichloroethane, dichloromethane, toluene, chlorobenzene, dichlorobenzene, acetone, diethyl ketone, iso-butyl methyl ketone, methyl acetate, ethyl acetate, n-propyl acetate, iso-propyl acetate, tetrahydrofuran, 2-methyltetrahydrofuran, tert-butanol and mixtures thereof.

8. The process as claimed in claim 7, wherein the solvent is selected from the group consisting of acetonitrile, dichloroethane, dichloromethane, toluene, acetone, ethyl acetate, iso-propyl acetate, tetrahydrofuran, 2-methyltetrahydrofuran, and mixtures thereof.

9. The process as claimed in claim 1, wherein the benzoxazinone of Formula II is converted to the compound of Formula I by in situ reaction of the benzoxazinone of Formula II with methylamine.

10. The process as claimed in claim 9, wherein the methylamine is aqueous or alcoholic methylamine solution.

11. A process for preparation of a compound of Formula I 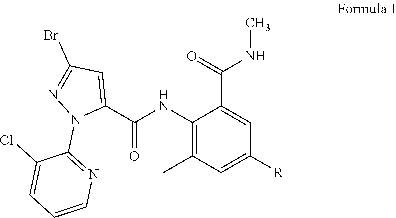
wherein R is Cl or CN
which comprises:
combining a pyrazolecarboxylic acid of Formula III, an anthranilic acid of Formula IV, a sulfonyl chloride and a base in a solvent to form a reaction mixture; 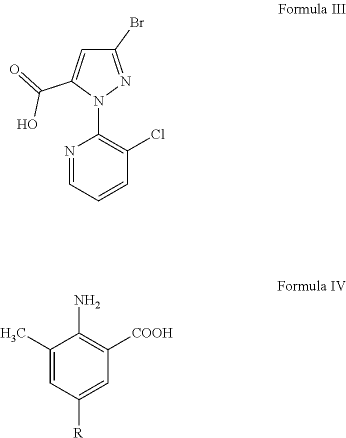
wherein R is Cl or CN
allowing the reaction mixture to react to form a benzoxazinone of Formula II; 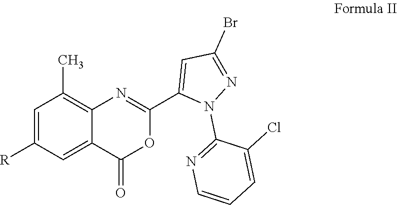
wherein R is Cl or CN
filtering the reaction mixture to yield a wet filter-cake comprising the benzoxazinone of Formula II; and
telescoping the wet filter-cake comprising the benzoxazinone of Formula II with methylamine to produce the compound of Formula I.

12. The process as claimed in claim 11, wherein the sulfonyl chloride is methanesulfonyl chloride, benzenesulfonyl chloride or p-toluenesulfonyl chloride.

13. The process as claimed in claim 11, wherein the base is an inorganic or organic base.

14. The process as claimed in claim 13, wherein the base is an inorganic base selected from the group consisting of sodium bicarbonate, sodium carbonate, potassium bicarbonate and potassium carbonate.

15. The process as claimed in claim 13, wherein the base is an organic base selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), triethylamine-DMAP, DABCO, N,N-dimethylaniline, and 4-(N,N-dimethyl)pyridine.

16. The process as claimed in claim 13, wherein the base is selected from the group consisting of 3-picoline, N-methylimidazole, 4-dimethylaminopyridine (DMAP), triethylamine-DMAP, sodium acetate and potassium carbonate.

17. The process as claimed in claim 11, wherein the solvent is selected from the group consisting of acetonitrile, dichloroethane, dichloromethane, toluene, chlorobenzene, dichlorobenzene, acetone, diethyl ketone, iso-butyl methyl ketone, methyl acetate, ethyl acetate, n-propyl acetate, iso-propyl acetate, tetrahydrofuran, 2-methyltetrahydrofuran, tert-butanol, and mixtures thereof.

18. The process as claimed in claim 17, wherein the solvent is selected from the group consisting of acetonitrile, dichloroethane, dichloromethane, toluene, acetone, ethyl acetate, iso-propyl acetate, tetrahydrofuran, 2-methyltetrahydrofuran, and mixtures thereof.

19. The process as claimed in claim 11, wherein the methylamine is aqueous or alcoholic methylamine solution.

20. A process for preparation of a benzoxazinone of Formula II 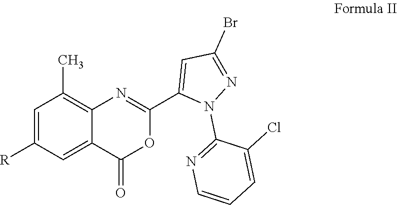
wherein R is Cl or CN
which comprises: reacting a pyrazolecarboxylic acid of Formula III, an anthranilic acid of Formula IV and a sulfonyl chloride in the presence of an inorganic base or an organic base 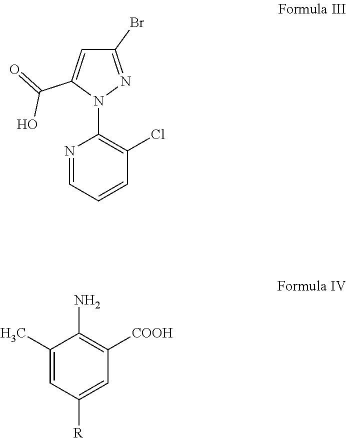
wherein R is Cl or CN
wherein the inorganic base is selected from the group consisting of sodium bicarbonate, sodium carbonate, potassium bicarbonate and potassium carbonate; and
wherein the organic base is selected from the group consisting of 2-picoline, 3-picoline, 2,6-lutidine, 3,4-lutidine, 2,4,6-collidine, diisopropylamine, diisopropylethylamine, triethylamine, trimethylamine, sodium acetate, N-methylmorpholine, N-methylpyrrolidine, N-methylimidazole, 2-methylimidazole, 4-dimethylaminopyridine (DMAP), triethylamine-DMAP, DABCO, N,N-dimethylaniline, and 4-(N,N-dimethyl)pyridine.

21. The process as claimed in claim 20, wherein the sulfonyl chloride is methanesulfonyl chloride, benzenesulfonyl chloride or p-toluenesulfonyl chloride.
